(19) United States
(12) Patent Application Publication
(10) Pub. No.: US 2023/0162825 A1
(43) Pub. Date: May 25, 2023

(54) HEALTH DATA PLATFORM AND ASSOCIATED METHODS

(71) Applicant: Truveta, Inc., Bellevue, WA (US)

(72) Inventors: Jayaram Nanduri, Issaquah, WA (US); Oscar Papel, Redmond, WA (US); Ramesh Kolavennu, Sammamish, WA (US); Ram Prasad Sunkara, Sammamish, WA (US); Terry Myerson, Issaquah, WA (US); George Joy, Kirkland, WA (US); Srinivasa R. Burugapalli, Sammamish, WA (US); Ryan Ahern, Seattle, WA (US)

(21) Appl. No.: 18/053,504

(22) Filed: Nov. 8, 2022

Related U.S. Application Data
(60) Provisional application No. 63/263,725, filed on Nov. 8, 2021.

(51) Int. Cl. G16H 10/60; G16H 40/20

(52) U.S. Cl. CPC G16H 10/60; G16H 40/20

(57) ABSTRACT

Systems and methods for processing and aggregating health data are disclosed herein. In some embodiments, a method for aggregating health data from a plurality of health systems includes receiving a set of patient records from a health system, and processing the set of patient records. The receiving and processing can be performed at an intermediary zone of a health data platform. The processing can include: (1) converting each patient record into a uniform format, and (2) generating a set of de-identified records from the set of patient records. The method can further include transmitting the set of de-identified records from the intermediary zone to a common data repository of the health data platform. The common data repository can be configured to store de-identified records from a plurality of different health systems.

### RELATED APPLICATIONS

This application claims the benefit of U.S. Provisional Pat. Application No. 63/263,725, entitled HEALTH DATA PLATFORM AND ASSOCIATED METHODS, filed on Nov. 8, 2021, which is herein incorporated by reference in its entirety. This application is related to U.S. Provisional Pat. Application No. 63/263,733, entitled “SYSTEMS AND METHODS FOR INDEXING AND SEARCHING HEALTH DATA,” filed on Nov. 8, 2021, U.S. Provisional Pat. Application No. 63/263,731, entitled “SYSTEMS AND METHODS FOR DE-IDENTIFYING PATIENT DATA,” filed on Nov. 8, 2021, U.S. Provisional Pat. Application No. 63/263,735, entitled “SYSTEMS AND METHODS FOR DATA NORMALIZATION,” filed on Nov. 8, 2021, U.S. Provisional Pat. Application No. 63/268,995, entitled “SYSTEMS AND METHODS FOR INDEXING AND SEARCHING HEALTH DATA,” filed on Mar. 8, 2022, U.S. Provisional Pat. Application No. 63/268,993, entitled “SYSTEMS AND METHODS FOR QUERYING HEALTH DATA,” filed on Mar. 8, 2022, U.S. Pat. Application No. ______, entitled “SYSTEMS AND METHODS FOR INDEXING AND SEARCHING HEALTH DATA,” filed concurrently herewith, U.S. Pat. Application No. ______, entitled “SYSTEMS AND METHODS FOR DE-IDENTIFYING PATIENT DATA,” filed concurrently herewith, U.S. Pat. Application No. ______, entitled “SYSTEMS AND METHODS FOR DATA NORMALIZATION,” filed concurrently herewith, each of which is herein incorporated by reference in its entirety.

### TECHNICAL FIELD

The present technology generally relates to healthcare, and in particular, to systems and methods for processing and aggregating data from multiple health systems.

### BACKGROUND

Healthcare entities such as hospitals, clinics, and laboratories produce enormous volumes of health data. This health data can provide valuable insights for research and improving patient care. However, the disclosure and use of certain types of health data are strictly limited by regulations and accepted practices. For example, the Health Insurance Portability and Accountability Act (HIPAA) Privacy Rule imposes stringent protections on protected health information (PHI), defined as individually identifiable health information that is held or transmitted by a HIPAA-covered entity (e.g., healthcare providers, insurers, healthcare clearinghouses) or business associate (e.g., a person or organization that provides certain services to a covered entity). Breaches of PHI can have serious implications on the lives of affected patients, can damage the trust that patients have in their healthcare providers, and can result in severe financial and regulatory penalties for the parties responsible for the breach.

The HIPAA Privacy Rule does not restrict the use or disclosure of de-identified health information—health information that neither identifies nor provides a reasonable basis for identifying a patient or individual. Typically, each health system performs de-identification on the health data it produces. However, different health systems may implement different de-identification processes, such that de-identified data produced by one health system may not be easily joinable with de-identified data from other health systems because, for example, they are not stored in a uniform format. Accordingly, improved systems and methods for processing and aggregating health data from multiple health systems are needed.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a schematic diagram of a computing environment in which a health data platform can operate, in accordance with embodiments of the present technology.

FIG. 1B is a schematic diagram of a data architecture that can be implemented by a health data platform, in accordance with embodiments of the present technology.

FIG. 1C is a schematic diagram of a data architecture adapted for use with a health system having multiple care sites, in accordance with embodiments of the present technology.

FIG. 2 is a flow diagram illustrating a method for processing and aggregating health data from a plurality of health data providers, in accordance with embodiments of the present technology.

### DETAILED DESCRIPTION

The present technology relates to systems and methods for processing and aggregating health data. In some embodiments, for example, a method for aggregating health data from a plurality of health systems includes receiving a set of patient records from a health system, and processing the set of patient records. The receiving and processing can be performed at an intermediary zone of a health data platform. The processing can include: (1) converting each patient record into a uniform format, and (2) generating a set of de-identified records from the set of patient records. The method can further include transmitting the set of de-identified records from the intermediary zone to a common data repository of the health data platform. The common data repository can be configured to store de-identified records from a plurality of different health systems.

The systems and methods of the present technology can provide a safe platform for aggregating health data from multiple health systems and/or other health data providers, thus enhancing the value of the data for commercial and/or research applications such as business processes, reporting, product health, customer intelligence, and/or artificial intelligence and machine learning. In particular, the intermediary zones described herein can serve as isolated, secure domains for converting patient records from specific health systems into a uniform format suitable for aggregation with other records in the common data repository of the health data platform. To reduce the risk of privacy breaches and/or other security concerns, the data within each intermediary zone can remain isolated from data from other health systems until the de-identification process is completed and the data is safe to transfer out. Additionally, each intermediary zone can include interfaces that allow the corresponding health system to audit the processes and stored data within that zone, thus further enhancing trust between the health system and the health data platform. In some embodiments, the disclosed techniques provide a network-based patient data management method that acquires and aggregates patient information from various sources into a uniform or common format, stores the aggregated patient information, and notifies health care providers and/or patients after information is updated via one or more communication channels. In some cases, the acquired patient information may be provided by one or more users through an interface, such as a graphical user interface, that provides remote access to users over a network so that any one or more of the users can provide at least one updated patient record in real time, such as a patient record in a format other than the uniform or common format, including formats that are dependent on a hardware and/or software platform used by a user providing the patient information.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The headings provided herein are for convenience only and do not interpret the scope or meaning of the claimed present technology. Embodiments under any one heading may be used in conjunction with embodiments under any other heading.

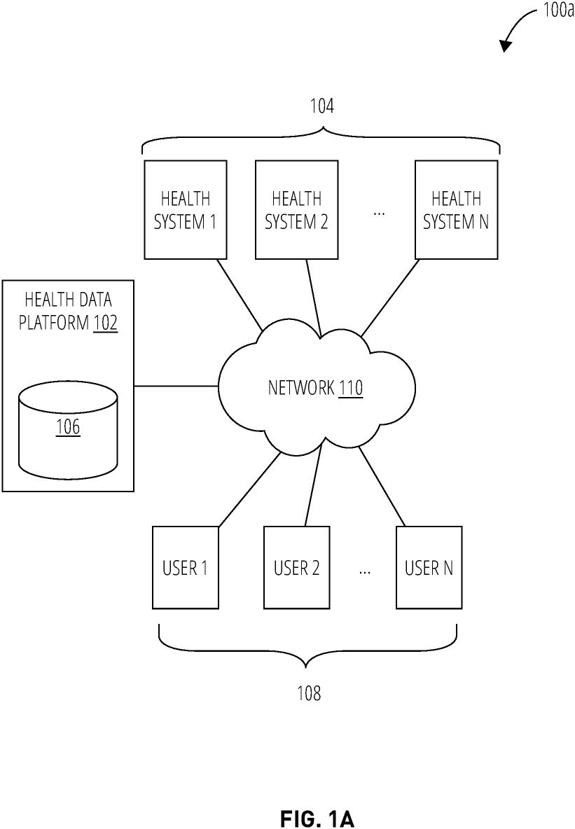

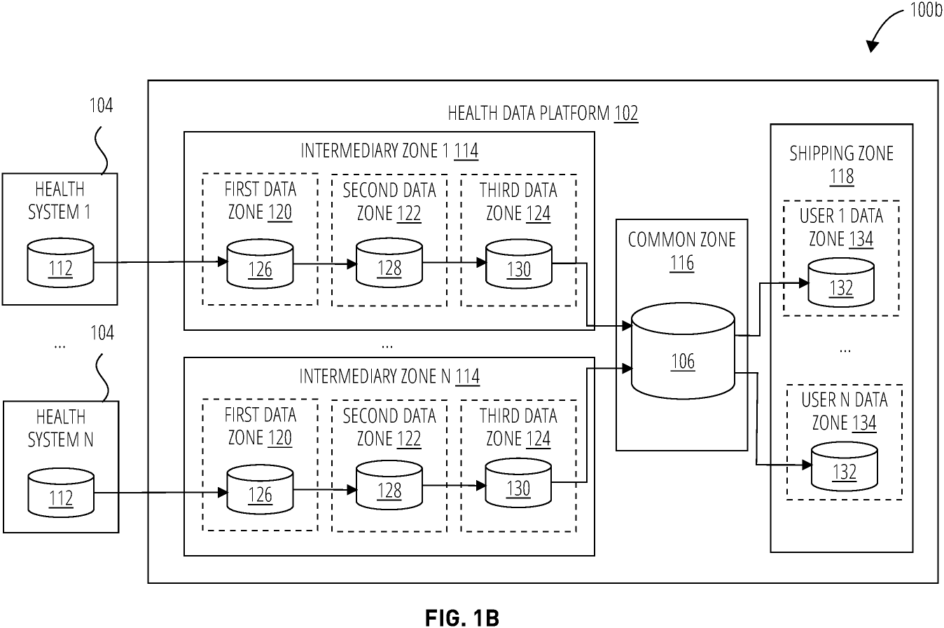

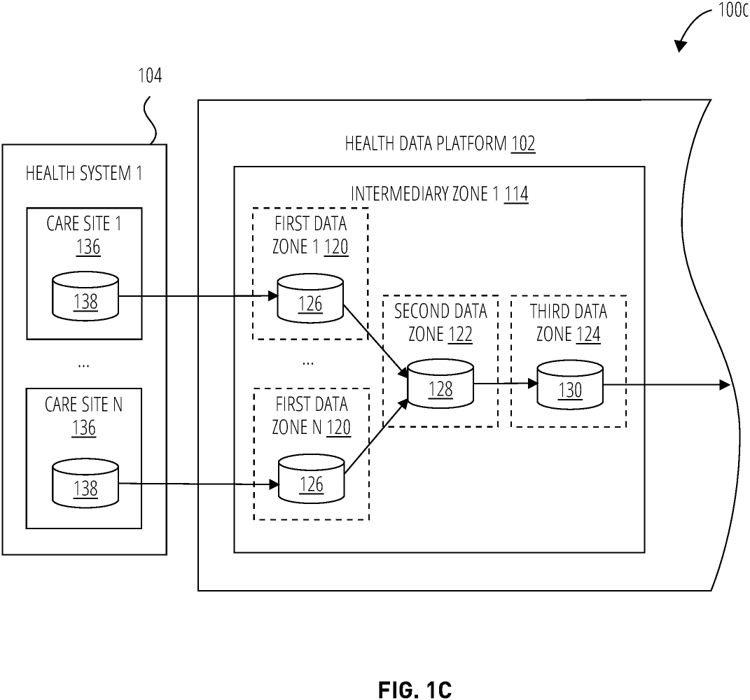

FIGS. 1A and 1B provide a general overview of a health data platform configured in accordance with embodiments of the present technology. Specifically, FIG. 1A is a schematic diagram of a computing environment 100a in which a health data platform 102 can operate, and FIG. 1B is a schematic diagram of a data architecture 100b that can be implemented by the health data platform 102.

Referring first to FIG. 1A, the health data platform 102 is configured to receive health data from a plurality of health systems 104, aggregate the health data into a common data repository 106, and allow one or more users 108 to access the health data stored in the common data repository 106. As described in further detail below, the common data repository 106 can store health data from multiple different health systems 104 and/or other data sources in a uniform schema, thus allowing for rapid and convenient searching, analytics, modeling, and/or other applications that would benefit from access to large volumes of health data.

The health data platform 102 can be implemented by one or more computing systems or devices having software and hardware components (e.g., processors, memory) configured to perform the various operations described herein. For example, the health data platform 102 can be implemented as a distributed “cloud” server across any suitable combination of hardware and/or virtual computing resources. The health data platform 102 can communicate with the health system 104 and/or the users 108 via a network 110. The network 110 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof.

The health data platform 102 can be configured to receive and process many different types of health data, such as patient data. Examples of patient data include, but are not limited to, the following: age, gender, height, weight, demographics, symptoms (e.g., types and dates of symptoms), diagnoses (e.g., types of diseases or conditions, date of diagnosis), medications (e.g., type, formulation, prescribed dose, actual dose taken, timing, dispensation records), treatment history (e.g., types and dates of treatment procedures, the healthcare facility or provider that administered the treatment), vitals (e.g., body temperature, pulse rate, respiration rate, blood pressure), laboratory measurements (e.g., complete blood count, metabolic panel, lipid panel, thyroid panel, disease biomarker levels), test results (e.g., biopsy results, microbiology culture results), genetic data, diagnostic imaging data (e.g., X-ray, ultrasound, MRI, CT), clinical notes and/or observations, other medical history (e.g., immunization records, death records), insurance information, personal information (e.g., name, date of birth, social security number (SSN), address), familial medical history, and/or any other suitable data relevant to a patient’s health. In some embodiments, the patient data is provided in the form of electronic health record (EHR) data, such as structured EHR data (e.g., schematized tables representing orders, results, problem lists, procedures, observations, vitals, microbiology, death records, pharmacy dispensation records, lab values, medications, allergies, etc.) and/or unstructured EHR data (e.g., patient records including clinical notes, pathology reports, imaging reports, etc.). A set of patient data relating to the health of an individual patient may be referred to herein as a “patient record.”

The health data platform 102 can receive and process patient data for an extremely large number of patients, such as thousands, tens of thousands, hundreds of thousands, millions, tens of millions, or hundreds of millions of patients. The patient data can be received continuously, at predetermined intervals (e.g., hourly, daily, weekly, monthly), when updated patient data is available and/or pushed to the health data platform 102, in response to requests sent by the health data platform 102, or suitable combinations thereof. Thus, due to the volume and complexity of the patient data involved, many of the operations performed by the health data platform 102 are impractical or impossible for manual implementation.

Optionally, the health data platform 102 can also receive and process other types of health data. For example, the health data can also include facility and provider information (e.g., names and locations of healthcare facilities and/or providers), performance metrics for facilities and providers (e.g., bed utilization, complication rates, mortality rates, patient satisfaction), hospital formularies, health insurance claims data (e.g., 835 claims, 837 claims), supply chain data (e.g., information regarding suppliers of medical devices and/or medications), device data (e.g., device settings, indications for use, manufacturer information, safety data), health information exchanges and patient registries (e.g., immunization registries, disease registries), research data, regulatory data, and/or any other suitable data relevant to healthcare. The additional health data can be received continuously, at predetermined intervals (e.g., hourly, daily, weekly, monthly), as updated data is available, upon request by the health data platform 102, or suitable combinations thereof.

The health data platform 102 can receive patient data and/or other health data from one or more health systems 104. Each health system 104 can be an organization, entity, institution, etc., that provides healthcare services to patients. A health system 104 can optionally be composed of a plurality of smaller administrative units (e.g., hospitals, clinics, labs, or groupings thereof), also referred to herein as “care sites.” The health data platform 102 can receive data from any suitable number of health systems 104, such as one, two, four, five, ten, fifteen, twenty, thirty, forty, fifty, hundreds, thousands, or more different health systems 104. Each health system 104 can include or otherwise be associated with at least one computing system or device (e.g., a server) that communicates with the health data platform 102 to transmit health data thereto. For example, each health system 104 can generate patient data for patients receiving services from the respective health system 104, and can transmit the patient data to the health data platform 102. As another example, each health system 104 can generate operational data relating to the performance metrics of the care sites within the respective health system 104, and can transmit the operational data to the health data platform 102.

Optionally, the health data platform 102 can receive health data from other data providers or data sources besides the health systems 104. For example, the health data platform 102 can receive health data from one or more databases, such as public or licensed databases on drugs, diseases, medical ontologies, demographics and/or other patient data, etc. (e.g., SNOMED CT, Rexnord, ICD-10, FHIR, LOINC, UMLS, OMOP, LexisNexis, state vaccine registries). In some embodiments, this additional health data provides metadata that is used to process, analyze, and/or enhance patient data received from the health systems 104, as described below.

The health data platform 102 can perform various data processing operations on the received health data, such as de-identifying health data that includes patient identifiers, converting the health data from a health system-specific format into a uniform format, and/or enhancing the health data with additional data. Subsequently, the health data platform 102 can aggregate the processed health data in the common data repository 106. The common data repository 106 can be or include one or more databases configured to store health data from multiple health systems 104 and/or other data sources. The health data in the common data repository 106 can be in a uniform schema or format to facilitate downstream applications. For example, the health data platform 102 can perform additional data processing operations on the health data in the common data repository 106, such as analyzing the health data (e.g., using machine learning models and/or other techniques), indexing or otherwise preparing the health data for search and/or other applications, updating the health data as additional data is received, and/or preparing the health data for access by third parties (e.g., by performing further de-identification processes). Additional details of some of the operations that can be performed by the health data platform 102 are described below with respect to FIG. 1B.

The health data platform 102 can allow one or more users 108 (e.g., researchers, healthcare professionals, health system administrators) to access the aggregated health data stored in the common data repository 106. Each user 108 can communicate with the health data platform 102 via a computing device (e.g., personal computer, laptop, mobile device, tablet computer) and the network 110. For example, a user 108 can send a request to the health data platform 102 to retrieve a desired data set, such as data for a population of patients meeting one or more conditions (e.g., diagnosed with a particular disease, receiving particular medication, belonging to a particular demographic group). The health data platform 102 can search the common data repository 106 to identify a subset of the stored health data that fulfills the requested conditions, and can provide the identified subset to the user 108. Optionally, the health data platform 102 can perform additional operations on the identified subset of health data before providing the data to the user, such as de-identification and/or other processes to ensure data security and patient privacy protection.

FIG. 1B illustrates the data architecture 100b of the health data platform 102, in accordance with embodiments of the present technology. The health data platform 102 can be subdivided into a plurality of discrete data handling zones, also referred to herein as “zones” or “domains.” Each zone is configured to perform specified data processing operations and store the data resulting from such operations. For example, in the illustrated embodiment, the health data platform 102 includes a plurality of intermediary zones 114 (also known as “embassies”) that receive and process health data from the health systems 104, a common zone 116 that aggregates the data from the intermediary zones 114 in the common data repository 106, and a shipping zone 118 that provides selected data for user access. Each zone can include a respective set of access controls, security policies, privacy rules, and/or other measures that define data isolation boundaries tailored to the sensitivity level of the data contained within that zone. The flow of data between zones can also be strictly controlled to mitigate the risk of privacy breaches and/or other data security risks.

The health data platform 102 is configured to receive data from a plurality of data providers, such as a plurality of health systems 104. Although certain embodiments herein are described with respect to receiving health data from health systems, the health data platform 102 can alternatively or additionally receive health data from other types of health data providers, such as insurance companies, government entities, research institutions, and/or any other organization or entity that may be a source of health data. The health data platform 102 can be operated by a service provider that is a separate entity or organization from the data providers. As described further below, the service provider can have administrative control over the entire health data platform 102, while the data providers can have audit access to and/or limited administrative capabilities over certain data zones within the health data platform 102.

In the illustrated embodiment, each of the health systems 104 includes at least one health system database 112. The health system database 112 can store health data produced by the respective health system 104, such as patient records containing data for patients receiving healthcare services from the health system 104, operational data for the health system 104, etc. The patient records stored in the health system database 112 can include or be associated with identifiers such as the patient’s name, address (e.g., street address, city, county, zip code), relevant dates (e.g., date of birth, date of death, admission date, discharge date), phone number, fax number, email address, SSN, medical record number, health insurance beneficiary number, account number, certificate or license number, vehicle identifiers and/or serial numbers (e.g., license plate numbers), device identifiers and/or serial numbers, web URL, IP address, finger and/or voice prints, photographic images, and/or any other characteristic or information that could uniquely identify the patient. Accordingly, the patient records can be considered to be PHI (e.g., electronic PHI (ePHI)), which may be subject to strict regulations on disclosure and use.

As shown in FIG. 1B, health data can be transmitted from the health systems 104 to the health data platform 102 via respective secure channels and/or over a communications network (e.g., the network 110 of FIG. 1A). The health data can be transmitted continuously, at predetermined intervals, in response to pull requests from the health data platform 102, when the health systems 104 push data to the health data platform 102, or suitable combinations thereof. For example, some or all of the health systems 104 can provide a daily feed of data to the health data platform 102.

The health data from the health systems 104 can be received by the intermediary zones 114 of the health data platform 102. Each intermediary zone 114 can be an independent system including a collection of software and services that are implemented in a suitable cloud platform (e.g., Microsoft Azure, Amazon Web Services, Google Cloud Platform). As shown in FIG. 1B, each intermediary zone 114 can be dedicated to a single respective health system 104, such that the intermediary zone 114 receives and processes health data from that health system 104 only. Different intermediary zones 114 can be isolated from each other such that health data across different health systems 104 cannot be combined with each other or accessed by unauthorized entities (e.g., a health system 104 other than the health system 104 that originated the data) before patient identifiers have been removed and/or the data is otherwise considered safe for aggregation.

In some embodiments, the intermediary zones 114 are configured to process the health data from the health systems 104 to prepare the data for export and aggregation in the common zone 116. For example, each intermediary zone 114 can de-identify the received health data to remove or otherwise obfuscate identifying information so that the health data is no longer classified as PHI and can therefore be aggregated and used in a wide variety of downstream applications (e.g., search, analysis, modeling). As another example, the intermediary zone 114 can also normalize the received health data by converting the data from a health system-specific format to a uniform format suitable for aggregation with health data from other health systems 104.

Each intermediary zone 114 can include a plurality of data zones or subsystems that sequentially process the health data from the respective health system 104. The data zones can be configured to store and process data having different sensitivity levels, e.g., one data zone can handle identifiable patient data, while another data zone can handle de-identified data. For example, in the illustrated embodiment, each intermediary zone 114 includes a first data zone 120 (also known as a “landing zone”), a second data zone 122 (also known as an “enhanced PHI zone”), and a third data zone 124 (also known as an “enhanced DeID zone”). As described in further detail below, the security policies, access controls, and/or other privacy protections implemented by each data zone can be customized to the data sensitivity level of that particular zone.

As shown in FIG. 1B, the health data (e.g., patient records) from each health system 104 can initially be received and processed by the first data zone 120 (landing zone). The first data zone 120 can implement one or more data ingestion processes to cleanse the data, such as by extracting relevant data and/or filtering out erroneous, corrupted, incomplete, and/or irrelevant data. The data ingestion processes can be customized based on the particular health system 104, such as based on the data architecture, data types, and/or data formats used by the health system 104. For example, the health system 104 can produce structured data, such as database tables and/or other data structures including schematized rows of data. Structured data can be organized by time, such as in the case of EHR fields related to temporal elements such as patient visits, treatments, measurements, prescriptions, or procedures. Structured data can be associated with schema definitions covering the set of fields, and the schema definitions can also be ingested along with the data itself. As another example, the health system 104 can produce unstructured or semi-structured data (e.g., in JSON format), such as clinical notes, documents scans, and reports. In a further example, the health system 104 can produce large files (e.g., image files, genomics data), which can be in custom file formats specific to the content type. Accordingly, the first data zones 120 within different intermediary zones 114 can implement different data ingestion processes, depending on the particular data type(s) produced by the corresponding health system 104.

The data resulting from the data ingestion processes (e.g., the patient records after data cleansing) can be stored in a first database 126 within the first data zone 120. The data can remain in the first database 126 indefinitely or for a limited period of time (e.g., no more than 30 days, no more than 1 year, etc.), e.g., based on the preferences of the respective health system 104, security considerations, and/or other factors. The data in the first database 126 can still be considered PHI because the patient identifiers have not yet been removed from the data. Accordingly, the first data zone 120 can be subject to relatively stringent access controls and data security measures.

In the illustrated embodiment, each health system 104 includes a single health system database 112 that serves as a unified data warehouse for all data produced by that health system 104. Accordingly, a single first data zone 120 may be sufficient for ingesting the data from the health system database 112. In other embodiments, however, a single health system 104 may include multiple independent or semi-independent data silos. For example, a health system 104 can include multiple care sites (e.g., smaller administrative units such as hospitals, clinics, labs, or groupings thereof), each of which may output different types and/or formats of health data. In such embodiments, it may be advantageous to implement different ingestion processes for receiving and/or cleansing the data from each care site.

FIG. 1C is a schematic diagram illustrating a data architecture 100c of the health data platform 102 that is adapted for use with a health system 104 having multiple care sites 136, in accordance with embodiments of the present technology. For example, the health system 104 can include two, three, four, five, ten, twenty, or more different care sites 136. Each care site 136 can include a respective care site database 138 storing health data produced by that care site 136. Some or all of the care sites 136 can produce health data having different types and/or data formats. Accordingly, the intermediary zone 114 for the health system 104 can include a plurality of first data zones 120 (landing zones), with each first data zone 120 configured to ingest data from a single respective care site 136. The data ingestion processes implemented by each first data zone 120 can be customized based on the data produced by the corresponding care site 136. The data resulting from the data ingestion processes can be stored in a respective first database 126 of each first data zone 120. The data can be combined in subsequent processes performed by the intermediary zone 114, as described further below.

Referring again to FIG. 1B, the data produced by the first data zone 120 can be transferred to the second data zone 122 (enhanced PHI zone). In embodiments where the intermediary zone 114 includes multiple first data zones 120 (FIG. 1C), the data from all the first data zones 120 can be transferred to and combined at a single second data zone 122. In some embodiments, the data received from the first data zone 120 is initially in a non-uniform format, such as a format specific to the health system 104 (or the care site 136 of the health system 104 (FIG. 1C)) that provided the data. Accordingly, the second data zone 122 can implement one or more data normalization processes to convert the data into a uniform, normalized format or schema, such as a standardized data model used for all data in the common data repository 106. Optionally, data normalization can also include enhancing, enriching, annotating, and/or otherwise supplementing the health data with additional data (e.g., health metadata received from databases and/or other data sources). The data normalization processes can evolve and/or otherwise be updated over time, e.g., to accommodate new downstream applications for the data such as machine learning and/or other advanced capabilities. Patient records that have undergone data normalization processes to convert the records into a uniform format and/or enhance the records with additional data may be referred to herein as “normalized records.”

The data resulting from these processes (e.g., normalized records) can be stored in a second database 128 within the second data zone 122. The data can remain in the second database 128 indefinitely or for a limited period of time (e.g., no more than 30 days, 1 year, etc.), e.g., based on the preferences of the respective health system 104, security considerations, and/or other factors. The data stored in the second database 128 can still be considered PHI because the patient identifiers have not yet been removed from the data. Accordingly, the second data zone 122 can also be subject to relatively stringent access controls and data security measures, similar to the first data zone 120.

The data produced by the second data zone 122 can be transferred to the third data zone 124 (enhanced DeID zone). The third data zone 124 can implement one or more de-identification processes to anonymize the data so that the data is no longer classified as PHI. For example, the third data zone 124 can generate de-identified records from the normalized records by removing, altering, coarsening, grouping, and/or shredding patient identifiers in the records, and/or removing or suppressing certain records altogether.

In some embodiments, the third data zone 124 applies a two-stage de-identification process that anonymizes the patient records while still allowing different records for the same patient to be matched and unified in downstream data zones. The first stage of the de-identification process can include generating tokens for each patient record, also referred to herein as “tokenization.” The tokens can be data elements that replace some or all of the identifiers in the patient record to serve as “fingerprints” to track an individual patient across the health data platform, but do not themselves contain any identifying information. In some embodiments, the tokens are used to identify different records in the health data platform that belong to the same patient, such as records for the same patient that are received at different times and/or are received from by different health systems 104. This approach allows the records to be matched and linked to each other to produce a single unified record for that patient, even after the records have been de-identified. In some embodiments, each token is generated from one or more identifiers in the patient record, such that the resulting token is unique to that patient (or has a high likelihood of being unique to that patient). The tokens can be generated using a suitable tokenization function, such as a cryptographic hash function.

The second stage of the de-identification process can include removing and/or modifying identifiers in each patient record, also referred to herein as “transformation.” The transformation process can eliminate, alter, and/or otherwise obfuscate some or all of the identifiers in each patient record so that the risk of the patient being re-identified from the remaining information in the transformed record is sufficiently small (e.g., based on k-anonymity and/or other anonymization standards known to those of skill in the art). For example, in some embodiments, the transformation process includes suppressing or redacting certain identifiers in each patient record (e.g., direct identifiers such as the patient’s name can be replaced with a placeholder character such as “*”). The transformation process can also include generalizing exact values or parameters in each record, such as by replacing them with broader ranges or categories (e.g., “10 years old” can be replaced with “1-18 years old” or “pediatric”; “Oregon” can be replaced with “Pacific Northwest”), or by coarsening them to reduce their level of specificity (e.g., a zip code of “98101” can be replaced with “98*”).

Optionally, the de-identification process can include suppressing certain patient records, even after they have been anonymized in accordance with the techniques described above. For example, a patient record can be suppressed if the record would still potentially be identifiable even after the identifiers have been removed and/or modified (e.g., if the record shows a diagnosis of an extremely rare disease).

The resulting de-identified data can be stored in a third database 130 within the third data zone 124. In some embodiments, it is important that de-identified records are stored separately from the original PHI-containing data (e.g., the patient records stored in the first database 126 and/or the normalized records stored in the second database 128) to prevent re-identification by using tokens to look up PHI. The data can remain in the third database 130 indefinitely or for a limited period of time (e.g., no more than 30 days, 1 year, etc.), e.g., based on the preferences of the respective health system 104, security considerations, and/or other factors. Because the data stored in the third database 130 is no longer considered PHI, the third data zone 124 can have less stringent access controls and data security measures than the first and second data zones 120, 122.

The de-identified data produced by each intermediary zone 114 can be transferred to a common zone 116 within the health data platform 102 via respective secure channels. The common zone 116 can include the common data repository 106 that stores aggregated de-identified data from all of the health systems 104. In some embodiments, the de-identified data still retains the identity of the health system 104 that originated that data, which can be advantageous for allowing for bulk data deletion of data produced by a particular health system 104 if that health system 104 withdraws from the health data platform 102, for ensuring that health system-specific use case prohibitions are honored, for calculating licensing royalties that are dependent on the nature and/or quality of the data, etc.

As discussed above, the data stored in the common data repository 106 has been de-identified and/or normalized into a uniform format, and can therefore be used in many different types of downstream applications. For example, the common zone 116 can implement processes that analyze the data in the common data repository 106 using machine learning and/or other techniques to produce various statistics, analytics (e.g., cohort analytics, time series analytics), models, knowledge graphs, etc. As another example, the common zone 116 can implement processes that index the data in the common data repository 106 to facilitate search operations.

The data stored in the common data repository 106 can be selectively transferred to the shipping zone 118 of the health data platform 102 for access by one or more users 108 (not shown in FIG. 1B). In the illustrated embodiment, the shipping zone 118 includes a plurality of user data zones 134. Each user data zone 134 can be customized for a particular user 108, and can store and expose a selected subset of data for access by that user 108 (e.g., via application programming interface (API) and/or terminal). The user data zones 134 can be isolated from each other so that each user 108 can only access data within their assigned user data zone 134. The amount, type, and/or frequency of data transferred to each user data zone 134 can vary depending on the data requested by the user 108 and the risk profile of the user 108. For example, the user 108 can send a request (e.g., a search query) to the health data platform 102 (e.g., via the network 110 of FIG. 1A) for access to certain data in the common data repository 106 (e.g., data for patients who have been diagnosed with a particular disease, belong to a particular population, have received a particular treatment procedure, etc.). The common zone 116 can implement a search process to determine a subset of the data in the common data repository 106 that fulfills the request parameters. Optionally, depending on the risk profile of the user 108, the common zone 116 can perform additional de-identification processes and/or apply other security measures to the data subset. The data subset can then be transferred to the user data zone 134 for access by the user 108 (e.g., via a secure channel in the network 110 of FIG. 1A).

As previously discussed, the data stored in the common data repository 106 of the common zone 116 can be in the same uniform, normalized schema as the data in the second and third data zones 122, 124 of the intermediary zones 114. Accordingly, any query that is executed on the data in the common zone 116 can also be executed on the data in the second and third data zones 122, 124, and vice-versa, also known as “query portability.” Query portability allows data from an individual health system 104 to be compared to data from all the health systems 104 linked to the health data platform 102, e.g., for purposes of assessing how the health outcomes of the individual health system 104 compare to the health outcomes across all the health systems 104. For example, a first search result can be obtained by executing a query on a set of patient records stored in an intermediary zone 114 associated with an individual health system 104, and a second search result can be obtained by executing the same query on the patient records stored in the common data repository 106. The first and second results can be compared to identify similarities, differences, trends, patterns, etc., in the patient records of the individual health system 104 versus the patient records across all the health systems 104.

In some embodiments, the common zone 116 receives and stores other types of data besides de-identified records from the intermediary zones 114. For example, the common zone 116 can also store machine learning models that can be applied to the data in the common data repository 106. The machine learning models can be provided by the health data platform 102 and/or by other data providers (e.g., researchers or other community members). Optionally, the common zone 116 can allow users to access and apply the stored machine learning models to their data.

As previously described, access to the various data handling zones of the health data platform 102 can be strictly controlled to ensure data security and compliance with the policies of the health systems 104, privacy standards, and/or other applicable regulations. In some embodiments, personnel associated with the service provider operating the health data platform 102 have access to and administrative privileges over the entire health data platform 102, but other individuals and/or entities have more restricted access. For example, personnel of a health system 104 can have access to and, optionally, limited administrative privileges over, the intermediary zone 114 for that health system 104 only, and not the intermediary zones 114 of any other health systems 104, the common zone 116, or the shipping zones 118. Similarly, a user can have access to their assigned user data zone 134 only, and not to any of the intermediary zones 114 or the common zone 116. These access controls can be implemented using any suitable identity and access management system, such as Azure Active Directory.

To further enhance trust, the health data platform 102 can optionally allow each health system 104 to monitor how its data is being used. In some embodiments, for example, each intermediary zone 114 is configured with at least one audit interface that exposes access points (e.g., via API and/or terminal) to allow the corresponding health system 104 to review and/or exert limited administrative capabilities over the processes and data flows within its intermediary zone 114. Accordingly, each health system 104 can have visibility into how its data is received, processed, stored, accessed, etc., without having to be responsible for operating and managing the intermediary zone 114. For example, the audit interface can allow each health system 104 to view the data (e.g., patient records) that is being transferred from its health system database 112 to the first data zone 120 of the intermediary zone 114 and, optionally, control the amount, frequency, and/or types of data being transferred. The audit interface can also allow the health system 104 to view data produced from some or all of the data processing stages that occur in the intermediary zone 114, such as the normalized records produced in the second data zone 122 and/or the de-identified records produced in the third data zone 124. Alternatively or in combination, the audit interface can allow the health system 104 to view summary statistics of the data and/or data processing that occurs in the intermediary zone 114, such as volumes of different types of patient data, average processing delays over a time interval, etc. In some embodiments, the audit interface allows each health system 104 to review the entities that have access to the data within its intermediary zone 114, as well as the administrative capabilities associated to each entity (e.g., security groups, policies, role assignments). Optionally, each health system 104 can be allowed to assign access to and/or administrative privileges over certain portions of the intermediary zone 114 (e.g., the first data zone 120) to specified personnel within its organization.

The data architecture 100b illustrated in FIG. 1B can be configured in many different ways. For example, although the intermediary zones 114 are illustrated in FIG. 1B as having three data zones, in other embodiments, some or all of the intermediary zones 114 can include fewer or more data zones. Any of the zones illustrated in FIG. 1B can alternatively be combined with each other into a single zone, or can be subdivided into multiple zones. Any of the processes described herein as being implemented by a particular zone can instead be implemented by a different zone, or can be omitted altogether.

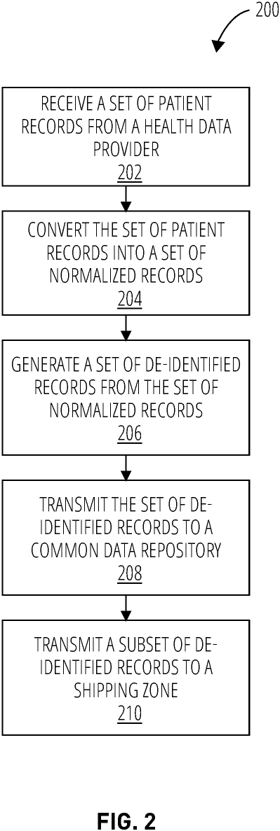

FIG. 2 is a flow diagram illustrating a method 200 for processing and aggregating health data from a plurality of health data providers, in accordance with embodiments of the present technology. The method 200 can be performed by any embodiment of the systems and devices described herein, such as by a computing system or device including one or more processors and a memory storing instructions that, when executed by the one or more processors, cause the computing system or the device to perform some or all of the steps described herein. For example, some or all of the steps of the method 200 can be performed by the health data platform 102 of FIGS. 1A-1C.

The method 200 begins at block 202 with receiving a set of patient records from a health data provider. As described above, the health data provider can be a health system, an individual care site of a health system, an insurance company, a government entity, a research institution, or any other organization or entity that supplies health data. In some embodiments, the process of block 202 includes receiving a large number of patient records, such as hundreds, thousands, tens of thousands, hundreds of thousands, millions, or tens of millions of patient records. Each patient record can include patient data for an individual patient, such as any of the patient data types described elsewhere herein (e.g., age, gender, height, weight, demographics, symptoms, diagnoses, medications, treatment history, vitals, laboratory measurements, test results, genetic data, diagnostic imaging data, clinical notes and/or observations, other medical history, insurance information, personal information, familial medical history, and the like). In some embodiments, each patient record includes one or more identifiers that can be used to identify that patient. These identifiers may need to be removed and/or modified before the patient record can be aggregated with other patient records from different health data providers.

In some embodiments, the patient records are received at a data handling zone or domain that is customized for that particular health data provider. For example, the patient records can be received at a first data zone 120 (landing zone) of a respective intermediary zone 114 of the health data platform 102 of FIG. 1B. As discussed above, the first data zone 120 can optionally implement an ingestion process to cleanse the received patient records, such as by extracting and/or filtering data.

At block 204, the method 200 can continue with converting the set of patient records into a set of normalized records. This process can include converting each patient record from a format specific to the health data provider to a uniform format or schema (e.g., a common format). The converting process can optionally include enhancing or otherwise supplementing the patient records with additional data. In some embodiments, the converting process of block 204 is performed by a different data zone than the data zone that received the patient records in block 202. For example, the converting can be performed by a second data zone 122 (enhanced PHI zone) of the intermediary zone 114 of the health data platform 102 of FIG. 1B.

At block 206, the method 200 can include generating a set of de-identified records from the set of normalized records. This process can include generating a plurality of tokens from identifiers in the normalized records, as well as removing and/or modifying some or all of the identifiers in the normalized records. In some embodiments, the de-identification process of block 206 is performed by a different data zone than the data zone that received the patient records in block 202 and/or the data zone that converted the patient records in block 204. For example, the converting can be performed by a third data zone 124 (enhanced DeID zone) of the intermediary zone 114 of the health data platform 102 of FIG. 1B.

At block 208, the method 200 can further include transmitting the set of de-identified records to a common data repository. The common data repository (e.g., the common data repository 106 of the common zone 116 of FIG. 1B) can be a unified data warehouse that stores aggregated de-identified records from a plurality of different health data providers (e.g., a plurality of different health systems). For example, the common data repository can store de-identified records from at least two, four, five, ten, fifteen, twenty, thirty, forty, fifty, or more different health data providers.

At block 210, the method 200 can include transmitting a subset of de-identified records from the common data repository to a shipping zone (e.g., the shipping zone 118 of FIG. 1B). The shipping zone can include a plurality of isolated data zones (e.g., the user data zones 134 of FIG. 1B) that selectively expose certain subsets of de-identified records for access by specific users. In some embodiments, the de-identified records are transferred to one of the user data zones 134 in response to a data access request from the corresponding user.

The method 200 illustrated in FIG. 2 can be modified in many different ways. For example, some or all of the steps of the method 200 can be repeated. In some embodiments, the health data provider provides a dynamic stream or feed of patient records to the health data platform, which may include records for new patients as well as updated records for existing patients. Accordingly, the processes of blocks 202-208 of the method 200 can be repeated (e.g., continuously, at predetermined intervals, when new data is available) to process and aggregate additional records from the same data provider. As another example, the processes of blocks 202-208 of the method 200 can be repeated for multiple health data providers to process and aggregate the records from each data provider. In such embodiments, the records from each data provider can remain isolated in the intermediary zone assigned to that provider until the records are processed and safe for aggregation with records from other data providers (e.g., after the de-identification process of block 206 is complete). Optionally, one or more of the steps of the method 200 can be omitted and/or the method 200 can include additional steps not shown in FIG. 2. As another example, method 200 may be modified to include one or more additional blocks, such as one or more blocks for automatically generating and transmitting messages to one or more users, such as a health care professional or patient. For example, in response to the health data platform receiving or acquiring new and/or updated records, the health data platform can de-identify the new and/or updated records, automatically generate a message containing the new and/or updated records whenever new and/or updated records are received or stored, and transmit the automatically generated message to one or more users over a network in real time, so that those users have immediate access to the new and/or updated patient records.

### EXAMPLES

The following examples are included to further describe some aspects of the present technology, and should not be used to limit the scope of the technology.

1. A computing system for aggregating health data from a plurality of health data providers, the system comprising:
one or more processors; and
a memory operably coupled to the one or more processors and storing instructions that, when executed by the one or more processors, cause the computing system to perform operations comprising:
   receiving, at an intermediary zone of a health data platform, a set of patient records from a health data provider;
   processing the set of patient records at the intermediary zone, wherein the processing includes: (1) converting the set of patient records into a set of normalized records, each normalized record having a uniform format, and (2) generating a set of de-identified records from the set of normalized records; and
   transmitting the set of de-identified records from the intermediary zone to a common data repository of a health data platform, wherein the common data repository is configured to store de-identified records from a plurality of different health data providers.

2. The system of Example 1, wherein the health data platform includes a plurality of intermediary zones, each intermediary zone being configured to receive data from a respective health data provider of the plurality of different health data providers.

3. The system of Example 2, wherein the plurality of intermediary zones are isolated from each other.

4. The system of Example 2 or Example 3, wherein each intermediary zone is customized for the respective health data provider.

5. The system of any one of Examples 1-4, wherein the receiving, converting, and generating occur over at least two different data zones within the intermediary zone.

6. The system of Example 5, wherein:
the receiving occurs at a first data zone within the intermediary zone;
the converting occurs at a second data zone within the intermediary zone; and
the generating occurs at a third data zone within the intermediary zone.

7. The system of Example 6, wherein:
the first data zone includes a first database configured to store the set of patient records;
the second data zone includes a second database configured to store the set of normalized records; and
the third data zone includes a third database configured to store the set of de-identified records.

8. The system of Example 6 or Example 7, wherein the health data provider is a health system including a plurality of care sites and the intermediary zone includes a plurality of first data zones, each first data zone configured to receive patient records from a corresponding care site.

9. The system of any one of Examples 1-8, wherein the set of patient records are received in a health system-specific format.

10. The system of any one of Examples 1-9, wherein the operations further comprise cleansing the set of patient records.

11. The system of any one of Examples 1-10, wherein converting the set of patient records into the set of normalized records comprises enhancing at least some of the patient records with additional data.

12. The system of any one of Examples 1-11, wherein each normalized record includes one or more patient identifiers, and generating the set of de-identified records comprises one or more of removing or modifying at least some of the patient identifiers in each normalized record.

13. The system of Example 12, wherein generating the set of de-identified records comprises producing one or more tokens from at least some of patient identifiers in each normalized record.

14. The system of any one of Examples 1-13, wherein each de-identified record in the common data repository is in the uniform format.

15. The system of any one of Examples 1-14, wherein the operations further comprise transmitting a subset of the de-identified records stored in the common data repository to a shipping zone.

16. The system of Example 15, wherein the shipping zone includes a plurality of user data zones, each user data zone configured for access by a respective user.

17. The system of any one of Examples 1-16, wherein the health data provider is a first health data provider, the intermediary zone is a first intermediary zone, and the operations further comprise:
receiving, at a second intermediary zone of the health data platform, a set of second patient records from a second health data provider;
processing the set of second patient records at the intermediary zone, wherein the processing includes: (1) converting the set of second patient records into a set of second normalized records, each second normalized record having the uniform format, and (2) generating a set of second de-identified records from the set of second normalized records; and
transmitting the set of second de-identified records from the second intermediary zone to the common data repository of the health data platform.

18. The system of Example 17, wherein the first intermediary zone is isolated from the second intermediary zone.

19. The system of any one of Examples 1-18, wherein the intermediary zone includes at least one audit interface configured to allow the health data provider to monitor the intermediary zone.

20. A method for aggregating health data from a plurality of health systems, the method comprising:
receiving, at an intermediary system of a health data platform, a set of patient records from a health system;
processing the set of patient records at the intermediary system, wherein the processing includes: (1) converting the set of patient records into a common format, and (2) generating a set of de-identified records from the set of patient records; and
transmitting the set of de-identified records from the intermediary system to a common data repository of the health data platform, wherein the common data repository is configured to store de-identified records from a plurality of different health systems.

21. The method of Example 20, wherein the health data platform includes a plurality of intermediary systems, each intermediary system being configured to receive data from a respective health system of the plurality of different health systems.

22. The method of Example 21, wherein the plurality of intermediary systems are isolated from each other.

23. The method of any one of Examples 20-22, wherein the intermediary system includes a plurality of data processing subsystems.

24. The method of Example 23, wherein the plurality of data processing subsystems includes:
a first data handling subsystem configured to receive the set of patient records;
a second data handling subsystem configured to convert the set of patient records into the common format; and
a third data handling subsystem configured to generate the set of de-identified records from the set of patient records.

25. The method of any one of Examples 20-24, further comprising:
receiving a request from a user to access a subset of the de-identified records in the common data repository; and
transmitting the subset of the de-identified records to a shipping zone separate from the common data repository.

26. The method of any one of Examples 20-25, further comprising providing an audit interface configured to allow the health system to monitor operations of the intermediary system.

27. The method of any one of Examples 20-26, further comprising:
generating a first search result by executing a query on the set of de-identified records at the intermediary system,
generating a second search result by executing the query on the de-identified records from the plurality of the different health systems stored in the common data repository, and
comparing the first and second search results.

28. The method of any one of Examples 1-27, further comprising:
storing, at the intermediary system of the health data platform, the set of patient records from the health system;
providing remote access to users over a network so that any one or more of the users can provide at least one updated patient record in real time through an interface, wherein at least one of the users provides an updated patient record in a format other than the common format, wherein the format other than the common format is dependent on hardware and software platform used by the at least one user;
converting the at least one updated patient record into the common format;
generating a set of at least one de-identified record from the at least one updated patient record;
storing, at the intermediary system, the generated set of at least one de-identified records;
after storing, at the intermediary system, the generated set of at least one de-identified record, generating a message containing the generated set of at least one de-identified record; and
transmitting the message to one or more users over the network in real time, so that the users have access to the updated patient record.

29. One or more non-transitory computer-readable storage media comprising instructions that, when executed by one or more processors of a computing system, cause the computing system to perform operations comprising:
receiving, at an intermediary zone of a health data platform, a set of patient records from a health system;
converting the set of patient records into a set of normalized records, each normalized record being in a uniform schema;
generating a set of de-identified records from the set of normalized records; and
transmitting the set of de-identified records from the intermediary zone to a common zone of the health data platform, wherein the common zone is configured to store de-identified records from a plurality of different health systems.

### Conclusion

Although many of the embodiments are described above with respect to systems, devices, and methods for processing patient data and/or other health data, the technology is applicable to other applications and/or other approaches. For example, the present technology can be used in other contexts where data privacy is an important consideration, such as financial records, educational records, political information, location data, and/or other sensitive personal information. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIG. 1A-2.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A computing system for aggregating health data from a plurality of health data providers, the system comprising:
one or more processors; and
a memory operably coupled to the one or more processors and storing instructions that, when executed by the one or more processors, cause the computing system to perform operations comprising:
receiving, at an intermediary zone of a health data platform, a set of patient records from a health data provider;
processing the set of patient records at the intermediary zone, wherein the processing includes: (1) converting the set of patient records into a set of normalized records, each normalized record having a uniform format, and (2) generating a set of de-identified records from the set of normalized records; and
transmitting the set of de-identified records from the intermediary zone to a common data repository of a health data platform, wherein the common data repository is configured to store de-identified records from a plurality of different health data providers.

2. The computing system of claim 1, wherein the health data platform includes a plurality of intermediary zones, each intermediary zone being configured to receive data from a respective health data provider of the plurality of different health data providers.

3. The computing system of claim 2, wherein the plurality of intermediary zones are isolated from each other.

4. The computing system of claim 2, wherein each intermediary zone is customized for the respective health data provider.

5. The computing system of claim 1, wherein the receiving, converting, and generating occur over at least two different data zones within the intermediary zone.

6. The computing system of claim 5, wherein:
the receiving occurs at a first data zone within the intermediary zone;
the converting occurs at a second data zone within the intermediary zone; and
the generating occurs at a third data zone within the intermediary zone.

7. The computing system of claim 6, wherein:
the first data zone includes a first database configured to store the set of patient records;
the second data zone includes a second database configured to store the set of normalized records; and
the third data zone includes a third database configured to store the set of de-identified records.

8. The computing system of claim 6, wherein the health data provider is a health system including a plurality of care sites and the intermediary zone includes a plurality of first data zones, each first data zone configured to receive patient records from a corresponding care site.

9. The computing system of claim 1, wherein the set of patient records are received in a health system-specific format.

10. The computing system of claim 1, wherein the operations further comprise cleansing the set of patient records.

11. The computing system of claim 1, wherein converting the set of patient records into the set of normalized records comprises enhancing at least some of the patient records with additional data.

12. The system of claim 1, wherein each normalized record includes one or more patient identifiers, and generating the set of de-identified records comprises one or more of removing or modifying at least some of the patient identifiers in each normalized record.

13. The computing system of claim 12, wherein generating the set of de-identified records comprises producing one or more tokens from at least some of patient identifiers in each normalized record.

14. The computing system of claim 1, wherein each de-identified record in the common data repository is in the uniform format.

15. The computing system of claim 1, wherein the operations further comprise transmitting a subset of the de-identified records stored in the common data repository to a shipping zone.

16. The computing system of claim 15, wherein the shipping zone includes a plurality of user data zones, each user data zone configured for access by a respective user.

17. The computing system of claim 1, wherein the health data provider is a first health data provider, the intermediary zone is a first intermediary zone, and the operations further comprise:
receiving, at a second intermediary zone of the health data platform, a set of second patient records from a second health data provider;
processing the set of second patient records at the intermediary zone, wherein the processing includes: (1) converting the set of second patient records into a set of second normalized records, each second normalized record having the uniform format, and (2) generating a set of second de-identified records from the set of second normalized records; and
transmitting the set of second de-identified records from the second intermediary zone to the common data repository of the health data platform.

18. The system of claim 1, wherein the intermediary zone includes at least one audit interface configured to allow the health data provider to monitor the intermediary zone.

19. A method for aggregating health data from a plurality of health systems, the method comprising:
receiving, at an intermediary system of a health data platform, a set of patient records from a health system;
processing the set of patient records at the intermediary system, wherein the processing includes: (1) converting the set of patient records into a common format, and (2) generating a set of de-identified records from the set of patient records; and
transmitting the set of de-identified records from the intermediary system to a common data repository of the health data platform, wherein the common data repository is configured to store de-identified records from a plurality of different health systems.

20. The method of claim 19, wherein the intermediary system includes a plurality of data processing subsystems, wherein the plurality of data processing subsystems includes:
a first data handling subsystem configured to receive the set of patient records;
a second data handling subsystem configured to convert the set of patient records into the common format; and
a third data handling subsystem configured to generate the set of de-identified records from the set of patient records.

21. The method of claim 19, further comprising:
receiving a request from a user to access a subset of the de-identified records in the common data repository; and
transmitting the subset of the de-identified records to a shipping zone separate from the common data repository.

22. The method of claim 21, further comprising:
generating a first search result by executing a query on the set of de-identified records at the intermediary system,
generating a second search result by executing the query on the de-identified records from the plurality of the different health systems stored in the common data repository, and
comparing the first and second search results.

23. The method of claim 19, further comprising:
storing, at the intermediary system of the health data platform, the set of patient records from the health system;
providing remote access to users over a network so that any one or more of the users can provide at least one updated patient record in real time through an interface, wherein at least one of the users provides an updated patient record in a format other than the common format, wherein the format other than the common format is dependent on hardware and software platform used by the at least one user;
converting the at least one updated patient record into the common format;
generating a set of at least one de-identified record from the at least one updated patient record;
storing, at the intermediary system, the generated set of at least one de-identified records;
after storing, at the intermediary system, the generated set of at least one de-identified record, generating a message containing the generated set of at least one de-identified record; and
transmitting the message to one or more users over the network in real time, so that the users have access to the updated patient record.

24. One or more non-transitory computer-readable storage media comprising instructions that, when executed by one or more processors of a computing system, cause the computing system to perform operations comprising:
receiving, at an intermediary zone of a health data platform, a set of patient records from a health system;
converting the set of patient records into a set of normalized records, each normalized record being in a uniform schema;
generating a set of de-identified records from the set of normalized records; and
transmitting the set of de-identified records from the intermediary zone to a common zone of the health data platform, wherein the common zone is configured to store de-identified records from a plurality of different health systems.
